(19) United States
(12) Patent Application Publication
(10) Pub. No.: US 2010/0130585 A1
(43) Pub. Date: May 27, 2010

(54) METHODS AND COMPOSITIONS FOR INVESTIGATION AND TREATMENT OF CANCER USING A G-PROTEIN COUPLED EICOSANOID RECEPTOR

(76) Inventors: Jagadananda Ghosh, Troy, MI (US); Sathish Sundaram, Ann Arbor, MI (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/063,842

(22) PCT Filed: Aug. 28, 2006
(86) PCT No.: PCT/US2006/033571

§ 371 Date: Feb. 14, 2008

Related U.S. Application Data
(60) Provisional application No. 60/711,607, filed on Aug. 26, 2005.

(51) Int. Cl. A61K 31/7088; C12Q 1/02; C07K 14/435; C07H 21/04; C12N 15/63; C12N 5/10; G01N 33/53; A61P 35/00

(52) U.S. Cl. 514/44 A; 435/29; 530/350; 536/23.5; 435/320.1; 435/325; 435/7.1

(57) ABSTRACT

In some embodiments, without limitation, the present invention comprises compositions and methods for the investigation and treatment of cancers by directly or indirectly blocking or interfering with the delivery of survival signals in cancer cells via a G protein-coupled receptor, in some embodiments, a 5-oxoETE or 5-HETE receptor. Some embodiments comprise the receptor itself and methods to manipulate, block, or interfere with expression of or activity by or through binding with the receptor, including without limitation, by interference with, inhibition, or prevention of action by metabolites of arachidonate 5-lipoxygenase, such as 5-HETE and its derivative, 5-oxoETE.

### CROSS-REFERENCE TO RELATED APPLICATIONS

This application claims priority based on U.S. Provisional Patent Application No. 60/711,607, filed Aug. 26, 2005, which is hereby incorporated by reference in full.

### GOVERNMENT GRANTS

This invention was made with government support of U.S. Department of Defense Prostate Cancer Research Programs, DAMD 17-01-1-0113, DAMD 17-02-1-0153, and W81XWH-05-1-0022. The government may have certain rights in the invention.

### FIELD OF THE INVENTION

This invention relates generally to the field of cancer therapeutics and treatments, including without limitation, compositions and methods for cancer treatments comprising novel therapeutic molecules.

### BACKGROUND OF THE INVENTION

Prostate cancer is the most common form of malignancy and second leading cause of cancer-related death in men in the United States [1]. Epidemiological studies and experiments with laboratory animals have repeatedly suggested a link between high-fat diets and clinical prostate cancer [2-6]. Biochemical and molecular studies have shown an association of fatty acid metabolism with prostate cancer cell proliferation. Specifically, metabolism of arachidonic acid has been shown to increase growth and promote survival of prostate cancer cells [7-13]. Arachidonic acid serves as substrate for several enzymes including lipoxygenases and cyclooxygenases. Metabolism of arachidonic acid by these enzymes generates a vast array of eicosanoids such as hydroxyeicosatetraenoids (HETEs), prostaglandins, and leukotrienes [14, 15]. (FIG. 1). These metabolites have been shown to influence growth and survival of a variety of cancer cells including that of the prostate [7, 8, 16]. 5-HETEs, generated by the action of 5-lipoxygenase (5-LOX), have been shown to be a principal arachidonate metabolite in prostate cancer cells [14, 16].

We have previously shown that inhibition of 5-LOX triggers apoptosis in prostate cancer cells. We have also shown that apoptosis triggered by 5-LOX inhibition could be prevented by exogenous 5(S)-hydroxyeicosatetraenoic acid (“5-HETE”) or its dehydroge-nated derivative 5-oxoeicosatetraenoic acid (“5-oxoETE”), and that 5-oxoETE was more effective in preventing apoptosis caused by 5-LOX inhibition [7, 8, 17]. Eicosanoids, such as the above arachidonic acid metabolites, have also been reported to play important roles in inflammatory and immune responses [18-24]. There are reports suggesting that these molecules act as signaling molecules in eosinophils and macrophages [18, 19]. For instance, 5-oxoETE has been shown to be an eosinophil chemoattractant [20]. Though 5-oxoETE plays an essential role in the survival of prostate cancer cells, its precise mechanism of action in the regulation of survival of these cancer cells is not yet fully elucidated.

Treatment options for prostate cancer, especially hormone refractory prostate cancer, are very limited. Thus, an unmet need remains for additional treatment options, particularly options that reflect current and developing understandings of the role of fatty acid metabolism in the etiology of prostate cancer and other pathologies.

### SUMMARY OF THE INVENTION

In some embodiments, without limitation, the invention comprises compositions and methods for the investigation and treatment of cancers by blocking the delivery of survival signals via a G-protein coupled cell 5-oxoETE receptor, designated “5-oxoER” (also “PCER”, for “prostate cancer eicosanoid receptor”). Some embodiments of the invention comprises the receptor itself and methods and compositions to manipulate, block, or interfere with expression of or activity by or through binding with the receptor, including without limitation, by interference with, inhibition, or prevention of action by metabolites of arachidonate 5-lipoxygenase, such as 5-HETE and its derivative, 5-oxoETE. Embodiments of the invention comprise a novel tool for the investigation and treatment of cancers, particularly human prostate cancer.

Fatty acid metabolism has been implicated in the survival and growth of a variety of cancer cell types. Metabolites of arachidonic acid (a common fatty acid in Western-style diets), especially 5-oxoETE, have been implicated in prostate cancer cell survival.

We have reported that metabolism of arachidonic acid through the 5-LOX pathway plays an important role in the survival and growth of human prostate cancer cells. Inhibition of 5-LOX by pharmacological inhibitors triggers apoptosis in prostate cancer cells within hours of treatment, which is prevented by the metabolites of arachidonate 5-lipoxygenase, 5-HETE, and its dehydrogenated derivative, 5-oxoETE. These findings suggested that 5-lipoxygenase metabolites are critical survival factors of prostate cancer cells. However, molecular mechanisms by which 5-HETE and its derivative 5-oxoETE exert their effects on prostate cancer cell survival are yet to be fully elucidated. (E.g., FIG. 1).

Our review of the literature determined a G-protein coupled receptor (GPCR) for which 5-oxoETE and to a lesser extent 5-HETE serve as agonists. Hosoi et al. and Jones et al. [25, 26] identified G protein-coupled receptors (TG1019 and R527, respectively) for which 5-oxoETE, and to a lesser extent 5-HETE, serve as ligands. Hosoi et al. [27] has also shown that the identified TG1019 receptor mediates 5-oxoETE-induced chemotaxis in CHO cells.

With our earlier observations on apoptosis caused by 5-LOX inhibition and its prevention by 5-oxoETE, we considered the novel possibility that a receptor of the 5-LOX metabolite, 5-oxoETE, is expressed in prostate cancer cells, and that this receptor plays a role in delivering survival signals of 5-oxo-ETE in these cells. In view of our earlier findings on 5-LOX inhibition-induced apoptosis and subsequent rescue with 5-oxoETE, we investigated the novel idea that 5-LOX metabolic products of arachidonic acid could serve as signaling molecules in prostate cancer cells. Using RT-PCR-based approach and gene knock-down experiments using siRNA, we checked for the expression of receptors for these molecules particularly, eicosanoid specific receptors, in prostate cancer cells.

We have discovered unexpectedly the expression of such an eicosanoid specific receptor in prostate cancer and the effects of interference with activity or expression of the 5-oxoETE receptor which comprise some embodiments of the present invention. We discovered unexpectedly that that a G protein-coupled receptor with high affinity for 5-oxoETE (designated “5-oxoER”) is highly expressed in prostate cancer cells. Our data also show that the more aggressive, hormone-refractory prostate cancer cells express higher levels of this receptor than the less aggressive hormone-sensitive cells. However, the expression of this receptor was undetectable in normal prostate epithelial cells in the same RT-PCR experiment.

We have isolated, cloned and sequenced this receptor from hormone-refractory prostate cancer cells. Moreover, our RNA-interference based experiments targeting this receptor in hormone-refractory prostate cancer cells clearly document its importance in prostate cancer cell survival, as well as as a direct or indirect target for therapies to alleviate, investigate, or prevent cancer. Blocking expression of 5-oxoER by short-interfering RNA (“siRNA”) significantly reduced the viability of prostate cancer cells, suggesting that 5-oxoER is critical for prostate cancer cell survival, and that the 5-LOX metabolite, 5-oxoETE, controls survival of prostate cancer cells through its own G protein-coupled receptor, 5-oxoER.

Thus, we have discovered the novel expression of an eicosanoid-specific receptor in prostate cancer and showed its importance in cancer cell survival. We report the expression of 5-oxoETE receptor in prostate cancer cells and show that it plays an important role in the survival of these cells, indicating that 5-oxoETE exerts its survival signal in prostate cancer cells through a receptor-mediated mechanism. Our discovery presents new therapeutic options for prostate cancer, including without limitation, hormone-refractory versions, as well as alternatives for investigating other therapeutics for such cancers.

### BRIEF DESCRIPTION OF THE DRAWINGS

The present invention will now be described, by way of example only and without limitation, with reference to the accompanying drawings, in which:

FIG. 1 is a diagram of postulated arachidonic acid signaling in prostate cancer survival.

FIG. 2 shows expression of 5-oxoETE receptor in two types of prostate cancer cells.

FIG. 3 shows the nucleotide and amino acid sequences of 5-oxoER isolated from PC3 prostate cancer cells.

FIGS. 4(A)-(B) show differential expression of 5-oxoER in prostate cancer cells.

FIGS. 5(A)-(B) show the effects of siRNA treatment on 5-oxoER gene expression and viability of prostate cancer cells.

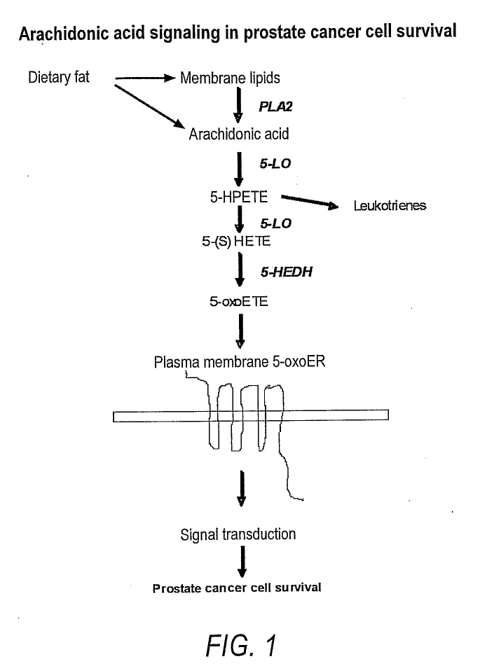

### DETAILED DESCRIPTION

In some embodiments, without limitation, the invention comprises compositions and methods for the investigation and treatment of cancers by blocking the delivery of survival signals via a G protein-coupled cell 5-oxoETE receptor, designated 5-oxoER. Some embodiments of the invention comprise the receptor itself as well as methods to manipulate, block, or interfere with expression of, activity by, or binding though the receptor, including without limitation, by interference with, inhibition, or prevention of action of metabolites of arachidonate 5-lipoxygenase, such as 5-HETE and its derivative, 5-oxoETE. In some preferred embodiments, without limiting the scope of the invention, the invention comprises a G protein-coupled 5-oxoETE receptor, designated 5-oxoER, in cancer cells, as one example only, mammalian prostate cancer cells, as well as compositions and methods preventing, inhibiting, or interfering with expression of or activity through the receptor, as one example only, through use of siRNA. Thus, some embodiments of the invention comprise novel compositions and tools for the treatment of cancers, particularly human prostate cancer.

Cell surfaces are known to contain specific receptors. Many biologically active substances function through interactions with those receptors, which are often coupled with a GTP-binding protein, or “G protein.” Those of ordinary skill in the art will appreciate that signals are transduced intracellularly via an activation of the G protein. Thus, many such receptors are known as “G protein-coupled receptors.”

In accordance with some embodiments of the invention, without limitation, we have isolated, cloned and sequenced a G protein-coupled receptor that is highly expressed in prostate cancer cells. We have discovered that the 5-lipoxygenase metabolite, 5-oxoETE, which is critical for prostate cancer cell survival, acts through this receptor. Our experiments with siRNA for this receptor confirm the novel finding that this receptor is essential for the survival of prostate cancer cells. This is the first report of the expression of this receptor in any cancer cell and of its critical role in the survival of prostate cancer cells. This makes this receptor a strong candidate for drug targeting.

In some embodiments, without limitation, the invention comprises SEQ ID NO: 1, a nucleotide sequence of a human cDNA of a gene of the G protein-coupled receptor isolated by us. SEQ ID NO: 2 represents an amino acid sequence of the receptor protein 5-oxoER encoded by that cDNA. The receptor protein comprising some embodiments of the invention is a G protein-coupled receptor protein which has a function and biological activity as a receptor of 5-oxoETE. As described herein, 5-oxoETE specifically binds to 5-oxoER, activating that receptor protein and inducing intracellular signaling as known to those of ordinary skill in the art.

In some embodiments, without limitation, the invention comprises a polypeptide having a function or an activity as a 5-oxoETE receptor, specifically, a polypeptide comprising SEQ ID NO: 2, and/or a polypeptide comprising an amino acid sequence shown by SEQ ID NO: 2 in which one or several amino acids are deleted, substituted or added. Embodiments of the invention also comprise, without limitation, a nucleic acid encoding the polypeptide(s); a recombinant vector and a host cell, comprising the above nucleic acid; a method of detecting a function or an activity of the polypeptide(s); a method for modulating (promoting or inhibiting) a function or an activity of such polypeptide(s); a method for screening or identifying a ligand or an effector (an agonist or an antagonist) of such polypeptide(s); a composition comprising as an effective ingredient an antagonist of such said polypeptide(s); and a method for treating and preventing cancer, comprising administering an effective amount of such an antagonist to a patient.

In our work, we have shown that expression of 5-oxoER in a cell or a tissue can be observed by typical immunological methods, as one example only, binding with specific labeled antibody. Those of ordinary skill in the art will appreciate that with the disclosure herein of the 5-oxoER polypeptide, it is possible to produce antibodies specific for regions of the polypeptide and affect the function or the activity of the receptor via an antibody directed against at least some portions of the polypeptide.

The 5-oxoER polypeptide of some embodiments functions as a 5-oxoETE receptor by specific binding to that molecule with concomitant induction of intracellular signaling as known to those of ordinary skill in the art. Such binding may be detected by, as one example only, use of a labeled ligand.

The 5-oxoER polypeptide can also be used to screen or identify ligands or effectors (e.g., agonists and/or antagonists) of the polypeptide. As one example only, and without limitation, the 5-oxoER polypeptide can be brought into contact with a test compound, and the degree and strength of specific binding, as well as the effect of such binding on induction of intracellular signals, can be assessed according to methods known to those of ordinary skill. In some embodiments, without limitation, optionally the 5-oxoER polypeptide can be used in a form of a membrane fraction or cells expressing the polypeptide on their surfaces.

As some examples, without limitation to only those disclosed, agonists of the receptor may be assessed to determine whether they induce intracellular signal transduction of a target cell or membrane bearing the 5-oxoER polypeptide. In corollary fashion, antagonists can be assessed, with or without the presence of an agonist like 5-oxoETE or other agonists, to determine whether the test compound inhibits the function or activity of the 5-oxoER polypeptide. In performing such assessments, appropriate controls may be utilized.

Antagonists of 5-oxoER can be used and administered in any pharmaceutically acceptable form. Consistent with our findings, administration of a 5-oxoER antagonist can induce apoptosis in prostate cancer cells. Therefore, by administering such a compound to a patient, it is possible to induce apoptosis in prostate cancer cells, and a pharmaceutical composition comprising a 5-oxo-ETE receptor antagonist as an active agent, and a method comprising a step of administering to a patient an effective amount of the 5-oxo-ETE receptor antagonist, can be applied in the treatment and prophylaxis of cancer.

Antagonists discovered in accordance with some embodiments of the invention, without limitation, may be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to, improved survival rate or more rapid recovery, or improvement or elimination of symptoms and other indicators as are selected as appropriate measures by those skilled in the art.

Such antagonists can be administered in various ways and are not limited to a particular way. It should be noted that they can be administered as the antagonist and can be administered alone or as an active ingredient in combination with pharmaceutically acceptable carriers, diluents, adjuvants and vehicles. The antagonists can be administered orally, subcutaneously or parenterally including intravenous, intraarterial, intramuscular, intraperitoneally, intranasal, transdermal or transmucosal administration, as well as intrathecal and infusion techniques.

Implants of the antagonists may also be also useful. The patient being treated is a warm-blooded animal and, in particular, mammals including humans. Pharmaceutically acceptable carriers, diluents, adjuvants and vehicles as well as implant carriers generally refer to inert, non-toxic solid or liquid fillers, diluents or encapsulating material not reacting with the active ingredients of the invention. The compound may be appropriately formulated into conventional preparations (tablets, granules, capsules, powders, inhalants, etc.) with an inert carrier depending on the administration route and used.

It is noted that humans are generally treated longer than experimental animals exemplified which treatment has a length proportional to the length of the disease process and drug effectiveness. The doses may be single doses or multiple doses over a period of several days. The treatment generally has a length proportional to the length of the disease process and drug effectiveness and the patient species being treated.

In some embodiments, without limitation, when administering the antagonist of the present invention parenterally, it may generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for, compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical four can be brought about by the use of agents delaying absorption, for example, aluminum monostearate and gelatin. According to the embodiments of present invention, however, any vehicle, diluent, or additive used would have to be compatible with the antagonists.

Sterile injectable solutions can be prepared by incorporating the antagonists utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the antagonists can be administered parenterally to the patient in the form of slow-release subcutaneous implants or targeted delivery systems such as monoclonal antibodies, vectored delivery, iontophoretic, polymer matrices, liposomes, and microspheres. Many other such implants, delivery systems, and modules are well known to those skilled in the art.

In one embodiment, the antagonist of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated.

The following examples describes some embodiment, of the invention, without limiting the scope of the invention or its embodiments to only those disclosed herein:

Cell culture and reagents. Androgen-sensitive (LNCaP) and androgen-refractory (PC3) human prostate cancer cells were obtained from American Type Culture Collection (Manassas, Va.). Cells were grown in RPMI medium 1640 (Invitrogen, Carlsbad, Calif.) supplemented with 10% fetal bovine serum (FBS) plus 100 U/ml penicillin and 100 μg/ml streptomycin. Cultures were maintained at 37 degrees C. in a humidified CO2-incubator. Cells were fed with fresh medium every third day and split at a confluence of about 80%. DNA primers for PCR were bought from Integrated DNA Technologies (Coralville, Iowa). SMARTpool siRNA against 5-oxoER was purchased from Dharmacon (Lafayette, Colo.). The lipid-based transfection reagent (TransIT-TKO) was purchased from Mirus (Madison, Wis.).

Expression and sequencing of 5-oxoETE receptor. Total RNA from LNCaP or PC3 cells was extracted using RNEasy mini kit (Qiagen, Valencia, Calif.). For RT-PCR, 25 ng of total RNA was used along with gene specific primers for 5-oxoETE receptor and GAPDH. First strand synthesis and amplification with gene specific primers were carried out with cMaster RT plus PCR system (Eppendorf, Westbury, N.Y.). The following primer pairs were used to test the expression of 5-oxoETE receptor in LNCaP and PC3 cells: (upstream) 5′-CAGTGGCTGCGAGAATGCTGATG-3′ (SEQ ID NO: 3) and downstream-5′-GGGAATGCCATCCTGGACAC-3′ (SEQ ID NO: 4) The 5-oxoETE receptor fragment containing the entire coding region was amplified using the primer set based on previously described method [25]. The coding region was then cloned into pcDNA3.1N5-His-TOPO vector (Invitrogen, Carlsbad, Calif.) and sequenced using BigDye Terminator V3.1 cycle sequencing kit and ABI PRISM 3700 sequencer (Applied Biosystems, Foster City, Calif.). Cloning and sequencing of the entire coding region of 5-oxoETE receptor was repeated (two independent experiments) to eliminate errors in sequencing.

Expression analysis. 25 ng total RNA each from LNCaP and PC3 cells were used for RT-PCR with 5-oxoER and GAPDH gene specific primers. PCR products were resolved by 1% agarose gel electrophoresis. Bands were analyzed with Eagle Eye II Darkroom Cabinet still video imaging system using EagleSightv3.1 software (Stratagene, La Jolla, Calif.). Band intensities of the receptor resolved in agarose gel were normalized to that of GAPDH.

siRNA treatment and assay of cell viability. PC3 prostate cancer cells (˜75,000 per well) were plated overnight onto 24-well plates in RPMI medium supplemented with 10% FBS. SMARTpool siRNA against 5-oxoER (with the following sense strand sequences: CAU GAG ACC UGG CGC UUU GUU, UCA CCU ACC UCA ACA GUG UUU, GGC GAG GUC UCU CUG GAA AUU, CAA AGU CAA CCU CUU CAU GUU) (SEQ ID NO: 5) was complexed with TransIT-TKO transfection reagent (Mirus) as per manufacturer's instructions and added directly to the media at varying concentrations. A bioinformatically designed siRNA (not targeting any known human or mouse gene) was used as control under the same experimental conditions (Dharmacon). Plates were incubated further for 72 h at 37° C. in the CO2 incubator. After isolation of total RNA, expression of 5-oxoER was detected and analyzed by RT-PCR using the primer set mentioned above. Expression of GAPDH was also measured in parallel as a control to verify the specific effect of siRNA treatment. For cell viability assays, PC3 cells were plated at about 3000 cells/well onto 96-well plates and treated with siRNA as mentioned above. Cell viability was measured by MTS/PES Cell Titer Aq assay (Promega, Madison, Wis.) as described previously [8].

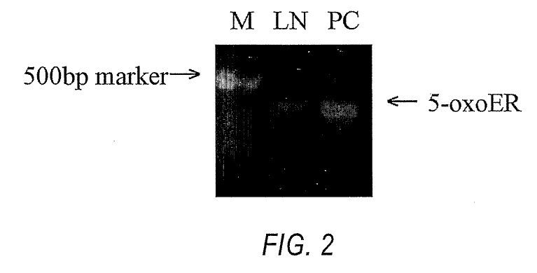

Prostate cancer cells express 5-oxoETE receptor. In accordance with some embodiments of the invention, without limitation, we examined whether human prostate cancer express a 5-oxoETE receptor (5-oxoER) by employing PCR-based analysis. We used gene specific primer sets for 5-oxoER (upstream, 5′-TCT TCA TCT TCT GCA TCC ACA CG-3′ (SEQ ID NO: 6) and (downstream) 5-AGT GGC AGG AAG AAC TCC AGC AG-3′ (SEQ ID NO: 7)) according to published procedure [26] to perform RT-PCR. Electrophoretic analysis of the PCR product showed a band of expected size (453 bp) in both androgen-sensitive (LNCaP) and androgen-refractory (PC3) human prostate cancer cells (FIG. 2). DNA sequencing of the PCR products revealed matches with a published sequence of a G protein coupled receptor [25, 26], confirming that human prostate cancer cells express 5-oxoETE receptor in them.

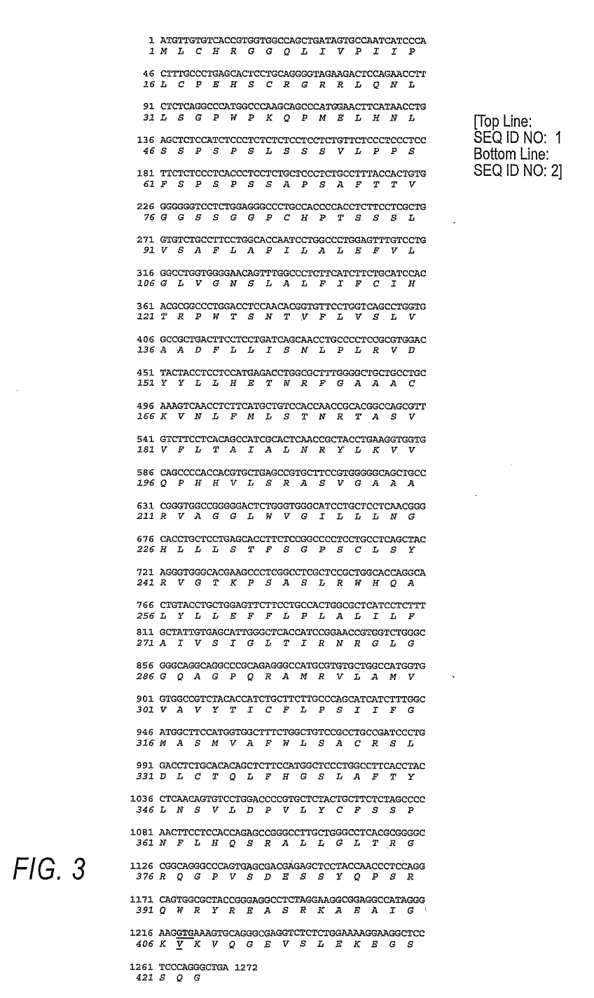

Cloning and sequencing of 5-oxoER. We cloned the complete sequence of the coding region of 5-oxoER in prostate cancer cells and compared it with the published TG1019 sequence in the database. We used primer sets flanking the coding region of the gene to isolate and clone the full length functional 5-oxoER from androgen-sensitive (LNCaP) and androgen-refractory (PC3) prostate cancer cells. Agarose gel analysis of the PCR product showed a band of about 1.5 kb which matched with the expected size of the 5-oxoER cDNA. We isolated the about 1.5 kb PCR product and cloned onto pcDNA3.1/V5-His-TOPO vector for sequencing. FIG. 3 shows the complete nucleic acid (and deduced amino acid sequences) of the coding region of 5-oxoER from PC3 prostate cancer cells (respectively, SEQ ID NO: 1 and SEQ ID NO: 2). The 5-oxoER was 423 amino acids in length and the nucleotide sequence was similar to that of TG1019 [25] except for a single nucleotide change (C to G) which corresponds to a single amino acid change from leucine to valine at position 407 (FIG. 3). This single amino acid difference was also observed in the 5-oxoETE receptor (R527) isolated by Jones et al. [26] except that R527 was only 384 amino acids in length PECR.

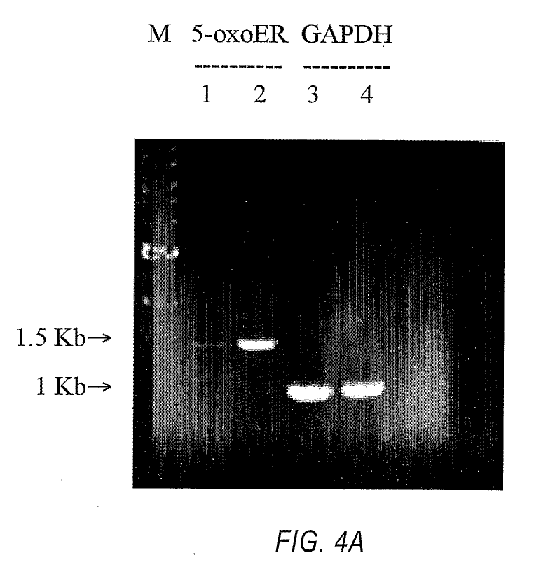

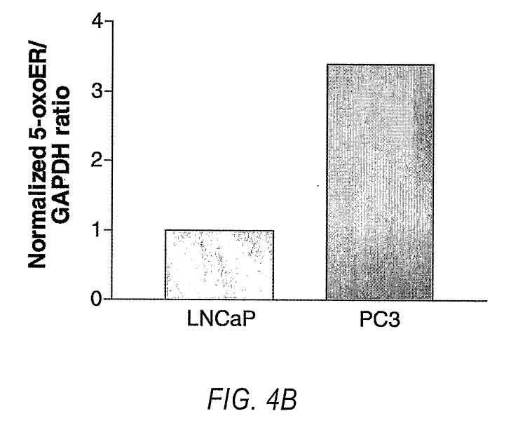

Androgen-sensitive (LNCaP) and androgen-refractory (PC3) prostate cancer cells differentially express 5-oxoETE receptor. To detect the relative levels of expression of 5-oxoER in androgen-sensitive and androgen-refractory prostate cancer cells, we used the same primer set (as in FIG. 3) covering the entire coding region of 5-oxoER for semi-quantitative RT-PCR experiments. Densitometric analysis of the PCR-generated product showed about 3.4-fold higher levels of the receptor in the androgen-refractory (and more aggressive) PC3 cells compared to the androgen-sensitive LNCaP cells (FIG. 4). FIG. 4 shows the differential expression of 5-oxoER in prostate cancer cells. FIG. 4(A) shows expression levels of 5-oxoER in androgen-sensitive (LNCaP) and androgen-refractory (PC3) prostate cancer cells were measured by RT-PCR using gene specific primers flanking the coding region. Lanes 1 and 3, LNCaP; lanes 2 and 4, PC3. Molecular weight standards are shown in lane M. FIG. 4(B) shows results from densitometric analysis of 5-oxoER expression in prostate cancer cells. Band densities of 5-oxoER and GAPDH were measured and the ratios were calculated for each cell type. 5-oxoER/GAPDH ratio for LNCaP was considered to be one unit of measurement. The normal prostate epithelial cells (“PrEC”) did not show any detectable expression of 5-oxoER under the same experimental conditions (not shown).

In order to determine whether the differential expression at RNA level is also reflected at the protein level, we performed western blot analyses with a polyclonal anti-rabbit IgG specific to the 5-oxoER. Our western blot analysis (not shown) also showed differential levels (about 3.4-fold difference between PC3 and LNCaP cells) of expression between hormone dependent and refractory cell lines consistent with the RT-PCR data.

We observed different levels of 5-oxoER expression in the androgen-sensitive (LNCaP) and the androgen-refractory (PC3) prostate cancer cells (FIG. 4). PC3 prostate cancer cells showed about 3.4-fold higher levels than LNCaP cells. LNCaP and PC3 cell lines were originally isolated from metastatic prostate cancer lesions of lymph node and bone, respectively. These two cell lines differ in a number of biological and molecular characteristics. Typically, LNCaP cells express androgen receptor, produce PSA (prostate-specific antigen), and have functional p53, whereas PC3 cells have none of these properties. Also, LNCaP cells have a longer generation time than PC3 cells and are much less tumorigenic in nude mice. Considering the growth-promoting and apoptosis-preventing effects of 5-oxoETE, it is likely that higher levels of expression of 5-oxoER in PC3 prostate cancer cells allow them to deliver stronger signals through 5-oxoER, and thus may contribute to their more aggressive growth and tumorigenic characteristics.

5-oxoER plays an important role in prostate cancer cell survival. Since we previously observed that 5-oxoETE plays a critical role in the survival of prostate cancer ells, we wanted to know whether its receptor (5-oxoER) also plays a similar role in the survival of these cells. In order to understand functional significance of 5-oxoER in prostate cancer cells, we studied the effect of its inhibition by gene silencing using siRNA to make these cells functional knockout of this receptor. We used androgen-refractory PC3 prostate cancer cells for siRNA treatment because of their higher level of expression of 5-oxoER.

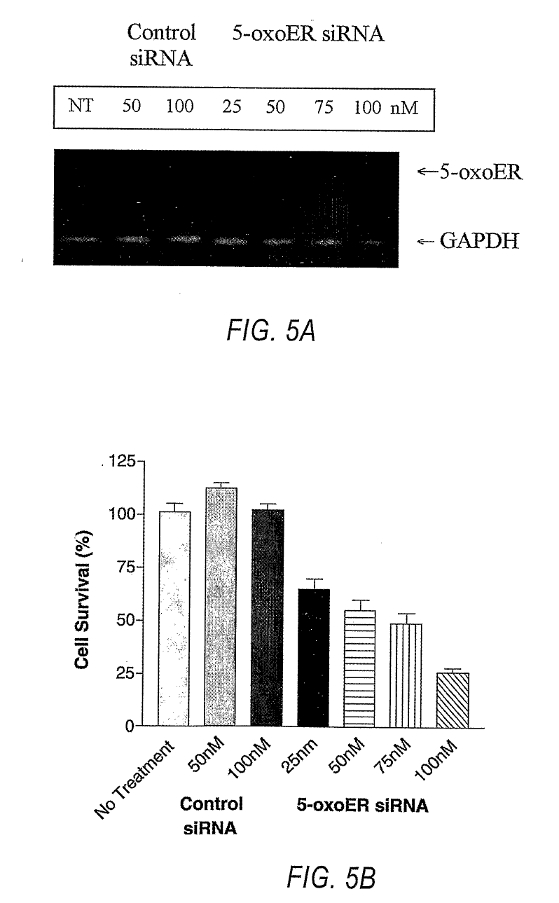

Our results showed a dose-dependent decrease in the expression of 5-oxoER in PC3 cells (FIG. 5A) and their viability (FIG. 5B) upon treatment with 5-oxoER siRNA. FIG. 5 shows the effects of siRNA treatment on 5-oxoER gene expression and viability of prostate cancer cells. PC3 prostate cancer cells (75,000 per well) were plated onto 24-well plates. Indicated concentrations of control (50, 100 nM) or 5-oxoER (25, 50, 75, and 100 nM) siRNA were added 24 h after seeding and the plates were incubated further for 7.2 h. NT represents no treatment. FIG. 5(A) shows expression levels of 5-oxoER detected by RT-PCR as in FIG. 2. Expression level of GAPDH was used as reference. FIG. 5(B) shows viability of PC3 cells 72 h after siRNA treatment. Absorbance values of untreated cells (no treatment) were designated to be 100% cell viability. Results are shown as mean values of each data point±standard error (n=3). These findings support our hypothesis that the growth-promoting and apoptosis-preventing effects of 5-oxoETE in prostate cancer cells are mediated through its receptor, 5-oxoER. In order to ensure that the observed effects were not a generic response to RNA interference, we used a bioinformatically designed control siRNA (not targeting any known human or mouse gene) in parallel under the same experimental conditions, which showed no significant effect on the expression of 5-oxoER, or the viability of cells.

We also measured the production of 5-oxoETE in PC3 prostate cancer cells by liquid chromatography/mass spectrometry “(LC/MS”) to assess whether prostate cancer cells constitutively produce 5-oxoETE as a survival factor. An inhibitor of 5-LOX, MK591, blocks production 5-oxoETE in PC3 (that also kills PC3 cells at this concentration). PC3 prostate cancer cells (2 million per plate) were plated in 6 ml RPMI medium in serum-free medium for 24 hours. On the next day cells were treated with 10 micromolar MK591 (a specific 5-LOX inhibitor) for two hours at 37 degree C., or cells were left untreated. The media were collected, eicosanoids extracted, and measured by LC/MS. Control PC3 cells displayed 11 picograms of eicosanoids per /ml of serum-free culture medium, while PC3 cells treated with MK591 (10 micromolar) showed 0 picograms per ml of serum-free culture medium. This evidence indicates that prostate cancer cells make 5-oxoETE constitutively.

Our data, for the first time, document the expression of 5-oxoER in human prostate cancer cells. Based on our previous findings about the essential role of 5-oxoETE in the survival and regulation of growth of prostate cancer cells [7, 8], and published reports about the possible existence of typical seven transmembrane G—protein-coupled receptors for this eicosanoid [28, 29], we hypothesized that 5-oxoETE may signal through its own G-protein-coupled receptor in regulating survival and growth of prostate cancer cells. Thus, we detected and characterized 5-oxoETE receptor gene expression through adopting a PCR based approach. G protein-coupled receptors of related eicosanoids, such as LTB4 and cysteinyl leukotrienes, have already been identified and characterized [30-36]. We were able to detect, clone, and sequence a 5-oxoETE receptor in human prostate cancer cells.

Our cDNA sequence of 5-oxoER in prostate cancer cells (FIG. 3) shows similarity to TG1019 [25] and suggests that it is a full length variant of TG1019 with a single nucleotide difference (C to G) which corresponds to a protein of 423 amino acids in length with a single amino acid change from leucine to valine at position 407. This single amino acid difference was also observed in the 5-oxoETE receptor (R527) isolated by Jones et al. [26]. However, the R527 receptor is only 384 amino acids in length (less 39 amino acids from the N-terminus).

Our siRNA treatment experiments with PC3 cells show that the receptor is critical for the survival of prostate cancer cells. This is consistent with previous findings that interference in the arachidonic acid metabolic pathway triggered cell death in these cells. 5-LOX metabolites of arachidonate have been previously implicated in prostate cancer progression, and the ability of 5-HETE and 5-oxoETE to rescue prostate cancer cells from 5-LOX inhibition induced apoptosis has been previously documented. Our findings indicate that these metabolites could be acting through the 5-oxoETE receptor (5-oxoER). The sensitivity of prostate cancer cells to treatment of siRNA against 5-oxoER suggest that the receptor is essential for the survival of prostate cancer cells and thus may be an appropriate therapeutic drug target for cancer treatment, particularly for prostate cancer.

As some examples only, compounds that directly or indirectly block or interfere with the binding of some products of arachidonate metabolism to the receptor decrease the survival-prolonging effects of such metabolites on cancer cells, resulting in their death. Compounds that block the expression of the receptor may have similar effects. Such compounds may comprise chemical or biological compounds or molecules, individually, in combination, or in mixture, and may be administered in dosages, methods of administration, and schedules known or determinable by those of ordinary skill in the art without undue experimentation. Embodiments of the invention thus include, without limitation, methods and compositions to manipulate, block, or interfere with expression of or activity by or through binding with the 5-oxoETE receptor, including without limitation, by interference with, inhibition, or prevention of action by metabolites of arachidonate 5-lipoxygenase, such as 5-HETE and its derivative, 5-oxoETE. Thus, some embodiments of the invention comprise novel compositions and methods for the investigation and treatment of cancers, particularly human prostate cancer.

Treatment of prostate cancer cells with siRNA against 5-oxoER indicates that the receptor plays an important role in the survival of these cells (FIG. 5). This is in direct correlation with our earlier observations that 5-oxoETE (the ligand of 5-oxoER) promotes prostate cancer cell growth and prevents apoptosis in these cells triggered by inhibition of arachidonate 5-lipoxygenase. Altogether, these findings suggest that 5-oxoETE exerts its pro-survival effects through 5-oxoER-mediated pathway. Downstream signaling mechanisms of 5-oxoETE and 5-oxoER in the regulation of prostate cancer cell growth and survival is not fully elucidated at this time. A role of 5-oxoER in mediating 5-oxoETE-induced chemotaxis has recently been characterized by Hosoi et al. transfecting CHO cells with 5-oxoER gene [27]. The expression of 5-oxoER in prostate cancer cells and its critical role in prostate cancer cell survival indicate that the growth-promoting and apoptosis-preventing effects of 5-oxoETE in prostate cancer cells are mediated through its cognate G-protein-coupled receptor, 5-oxoER, and that 5-oxoER comprises an appropriate molecular target for the treatment of prostate cancer.

This application references various publications by author, citation, and/or by patent number, including without limitation, articles, presentations, and United States patents. The disclosures of each of these references in their entireties are hereby incorporated by reference into this application.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the apparatus within the scope of these claims and their equivalents be covered thereby. This description of the invention should be understood to include all novel and non-obvious combinations of elements described herein, and claims may be present in this or a later application to any novel and non-obvious combination of these elements. The foregoing embodiments are illustrative, and no single feature or element is essential to all possible combination that may be claimed in this or a later application. Where the claims recite “a” or “a first” element of the equivalent thereof, such claims should be understood to include incorporation of one or more such elements, neither requiring nor excluding two or more such elements.

### REFERENCES

[1] A. Jemal, R. C. Tiwari, T. Murray, A. Ghafoor, A. Samuels, E. Ward, E. J. Feuer, M. J. Thun, Cancer Statistics, 2004, CA Cancer J. Clin. 54 (2004) 8-29.
[2] M. P. Porter, J. L. Stanford, Obesity and the risk of prostate cancer, Prostate 62 (2005) 316-321.
[3] J. H. Mydlo, The impact of obesity in urology, Urol. Clin. North Am. 31 (2004) 275-287.
[4] N. Fleshner, P. S. Bagnell, L. Klotz, V. Venkateswaran, Dietary fat and prostate cancer, J. Urol. 171 (2004) S19-S24.
[5] A. M. Kamat, D. L. Lamm, Diet and nutrition in urologic cancer, WV Med. J. 96 (2000) 449-454.
[6] P. Correa, Epidemiological correlations between diet and cancer frequency, Cancer Res. 41 (1981) 3685-3690.
[7] J. Ghosh, C. E. Myers, Inhibition of arachidonate 5-lipoxygenase triggers massive apoptosis in human prostate cancer cells, Proc. Natl. Acad. Sci. USA 95 (1998) 13182-13187.
[8] J. Ghosh, C. E. Myers, Arachidonic acid stimulates prostate cancer cell growth: critical role of 5-lipoxygenase, Biochem. Biophys. Res. Commun. 235 (1997) 418-423.
[9] X. Gao, D. J. Grignon, T. Chbihi, A. Zacharek, Y. Q. Chen, W. Sakr, A. T. Porter, J. D. Crissman, J. E. Pontes, I. J. Powell, Elevated 12-lipoxygenase mRNA expression correlates with advanced stage and poor differentiation of human prostate cancer, Urology 46 (1995) 227-237.
[10] J. Ghosh, C. E. Myers, Central role of arachidonate 5-lipoxygenase in the regulation of cell growth and apoptosis in human prostate cancer cells, Adv. Exp. Med. Biol. 469 (1999) 577-582.
[11] J. Ghosh, Inhibition of arachidonate 5-lipoxygenase triggers prostate cancer cell death through rapid activation of c-Jun N-terminal kinase, Biochem. Biophys. Res. Commun. 307 (2003) 342-349.
[12] D. Nie, M. Cher, D. Grignon, K. Tang, K. V. Honn, Role of eicosanoids in prostate cancer progression, Cancer Metastasis Rev. 20 (2001) 195-206.
[13] K. M. Anderson, T. Seed, M. Vos, J. Muishine, J. Meng, W. Alrefai, D. Ou, J. E. Harris, 5-Lipoxygenase inhibitors reduce PC-3 cell proliferation and initiate nonnecrotic cell death, Prostate 37 (1998) 161-173.
[14] S. H. Lee, M. V. Williams, R. N. DuBois, I. A. Blair, Targeted lipidomics using electron capture atmospheric pressure chemical ionization mass spectrometry, Rapid Commun. Mass Spectrom. 17 (2003) 2168-2176.
[15] F. Q. Wen, K. Watanabe, M. Yoshida, Eicosanoid profile in cultured human pulmonary artery smooth muscle cells treated with IL-1 beta and TNF alpha, Prostaglandins Leukot. Essent. Fatty Acids 59 (1998) 71-75.
[16] D. P. Rose, J. M. Connolly, Regulation of tumor angiogenesis by dietary fatty acids and eicosanoids, Nutr. Cancer 37 (2000) 119-127.
[17] J. Ghosh, Rapid induction of apoptosis in prostate cancer cells by selenium: reversal by metabolites of arachidonate 5-lipoxygenase, Biochem. Biophys. Res. Commun. 315 (2004) 624-635.
[18] R. Ramires, M. F. Caiaffa, A. Tursi, J. Z. Haeggstrom, L. Macchia, Novel inhibitory effect on 5-lipoxygenase activity by the anti-asthma drug montelukast, Biochem. Biophys. Res. Commun. 324 (2004) 815-821.
[19] L. Borish, B. Z. Joseph, Inflammation and the allergic response, Med. Clin. North Am. 76 (1992) 765-787.
[20] K. R. Erlemann, J. Rokach, W. S. Powell, Oxidative stress stimulates the synthesis of the eosinophil chemoattractant 5-oxo-6,8,11,14-eicosatetraenoic acid by inflammatory cells, J. Biol. Chem. 279 (2004) 40376-40384.
[21] S. P. Colgan, Lipid mediators in epithelial cell-cell interactions, Cell Mol. Life Sci. 59 (2002) 754-760.
[22] V. D. Pasechnikov, Y. A. Radsev, S. P. Guminskij, 5-Lipoxygenase products: their biosynthesis in human gastric mucosa and possible involvement in inflammatory response and oxygen saturation index reduction in gastric ulcer patients, Biochim. Biophys. Acta 1097 (1991) 45-48.
[23] J. L. Masferrer, J. A. Rimarachin, M. E. Gerritsen, J. R. Falck, P. Yadagiri, M. W. Dunn, M. Laniado-Schwartzman, 12(R)-hydroxyeicosatrienoic acid, a potent chemotactic and angiogenic factor produced by the cornea, Exp. Eye Res. 52 (1991) 417-424.
[24] R. Rochels, W. D. Busse, In vivo evidence for the chemotactic activity of cyclooxygenase- and lipoxygenase-dependent compounds using a corneal implantation technique, Ophthalmic Res. 16 (1984) 194-197.
[25] T. Hosoi, Y. Koguchi, E. Sugikawa, A. Chikada, K. Ogawa, N. Tsuda, N. Suto, S. Tsunoda, T. Taniguchi, T. Ohnuki, Identification of a novel human eicosanoid receptor coupled to G(i/o), J. Biol. Chem. 277 (2002) 31459-31465.
[26] C. E. Jones, S. Holden, L. Tenaillon, U. Bhatia, K. Seuwen, P. Tranter, J. Turner, R. Kettle, R. Bouhelal, S. Charlton, N. R. Nirmala, G. Jarai, P. Finan, Expression and characterization of a 5-oxo-6E,8Z,1 1Z,14Z-eicosatetraenoic acid receptor highly expressed on human eosinophils and neutrophils, Mol. Pharmacol. 63 (2003) 471-477.
[27] T. Hosoi, E. Sugikawa, A. Chikada, Y. Koguchi, T. Ohnuki, TG1019/OXE, a Galpha(i/o)-protein-coupled receptor, mediates 5-oxo-eicosatetraenoic acid-induced chemotaxis, Biochem. Biophys. Res. Commun. 334 (2005) 987-995.
[28] W. S. Powell, S. Gravel, R. J. MacLeod, E. Mills, M. Hashefi, Stimulation of human neutrophils by 5-oxo-6,8,11,14-eicosatetraenoic acid by a mechanism independent of the leukotriene B4 receptor, J. Biol. Chem. 268 (1993) 9280-9286.
[29] J. T. O˜Flaherty, J. S. Taylor, M. Kuroki, The coupling of 5-oxoeicosanoid receptors to heterotrimeric G proteins, J. Immunol. 164 (2000) 3345-3352.
[30] J. H. Kehrl, G-protein-coupled receptor signaling, RGS proteins, and lymphocyte function, Crit. Rev. Immunol. 24 (2004) 409-423.
[31] C. K. Nielsen, J. I. Campbell, T. F. Ohd, M. Morgelin, K. Riesbeck, G. Landberg, A. Sjolander, A novel localization of the G-protein coupled CysLT1 receptor in the nucleus of colorectal adenocarcinoma cells, Cancer Res. 65 (2005) 732-742.
[32] C. Corrigan, K. Mallett, S. Ying, D. Roberts, A. Parikh, G. Scadding, T. Lee, Expression of the cysteinyl leukotriene receptors cysLT(1) and cysLT(2) in aspirin-sensitive and aspirin-tolerant chronic rhinosinusitis, J. Allergy Clin. Immunol. 115 (2005) 316-322.
[33] C. K. Nielsen, R. Massoumi, M. Sonnerlind, A. Sjolander, Leukotriene D4 activates distinct O-proteins in intestinal epithelial cells to regulate stress fibre formation and to generate intracellular Ca²⁺ mobilisation and ERK1/2 activation, Exp. Cell Res. 302 (2005) 31-39.
[34] F. Sallusto, C. R. Mackay, Chemoattractants and their receptors in homeostasis and inflammation, Curt. Opin. Immunol. 16 (2004) 724-773.
[35] D. Mesnier, J. L. Baneres, Cooperative conformational changes in a G-protein-coupled receptor dimer, the leukotriene B (4) receptor BLT1, J. Biol. Chem. 279 (2004) 49664-49670.
[36] Z. Chen, R. Gaudreau, C. Le Gouill, M. Rola-Pleszczynski, J. Stankova, Agonist-induced internalization of leukotriene B (4) receptor 1 requires O-protein-coupled receptor kinase 2 but not arrestins, Mol. Pharmacol. 66 (2004) 377-386.
[37] A. van Bokhoven, M. Varella-Garcia, C. Korth, W. U. Johannes, E. E. Smith, H. L. Miller, S. K. Nordeen, G. J. Miller, M. S. Lucia, Molecular characterization of human prostate carcinoma cell lines, Prostate 57 (2003) 205-225.

| | |
|---|---|
| SEQ ID NO: 1: | |
| atgttgtgtc accgtggtgg ccagctgata gtgccaatca tcccactttg ccctgagcac | 60 |
| tcctgcaggg gtagaagact ccagaacctt ctctcaggcc catggcccaa gcagcccatg | 120 |
| gaacttcata acctgagctc tccatctccc tctctctcct cctctgttct ccctccctcc | 180 |
| ttctctccct caccctcctc tgctccctct gcctttacca ctgtgggggg gtcctctgga | 240 |
| gggccctgcc accccacctc ttcctcgctg gtgtctgcct tcctggcacc aatcctggcc | 300 |
| ctggagtttg tcctgggcct ggtggggaac agtttggccc tcttcatctt ctgcatccac | 360 |
| acgcggccct ggacctccaa cacggtgttc ctggtcagcc tggtggccgc tgacttcctc | 420 |
| ctgatcagca acctgcccct ccgcgtggac tactacctcc tccatgagac ctggcgcttt | 480 |
| ggggctgctg cctgcaaagt caacctcttc atgctgtcca ccaaccgcac ggccagcgtt | 540 |
| gtcttcctca cagccatcgc actcaaccgc tacctgaagg tggtgcagcc ccaccacgtg | 600 |
| ctgagccgtg cttccgtggg ggcagctgcc cgggtggccg ggggactctg ggtgggcatc | 660 |
| ctgctcctca acgggcacct gctcctgagc accttctccg gcccctcctg cctcagctac | 720 |
| agggtgggca cgaagccctc ggcctcgctc cgctggcacc aggcactgta cctgctggag | 780 |
| ttcttcctgc cactggcgct catcctcttt gctattgtga gcattgggct caccatccgg | 840 |
| aaccgtggtc tgggcgggca ggcaggcccg cagagggcca tgcgtgtgct ggccatggtg | 900 |
| gtggccgtct acaccatctg cttcttgccc agcatcatct ttggcatggc ttccatggtg | 960 |
| gctttctggc tgtccgcctg ccgatccctg gacctctgca cacagctctt ccatggctcc | 1020 |
| ctggccttca cctacctcaa cagtgtcctg gaccccgtgc tctactgctt ctctagcccc | 1080 |
| aacttcctcc accagagccg ggccttgctg ggcctcacgc ggggccggca gggcccagtg | 1140 |
| agcgacgaga gctcctacca accctccagg cagtggcgct accgggaggc ctctaggaag | 1200 |
| gcggaggcca tagggaaggt gaaagtgcag ggcgaggtct ctctggaaaa ggaaggctcc | 1260 |
| tcccagggct ga | 1272 |
| SEQ ID NO: 2: | |
| Met Leu Cys His Arg Gly Gly Gln Leu Ile Val Pro Ile Ile Pro Leu | |
| 1 5 10 15 | |
| Cys Pro Gln His Ser Cys Arg Gly Arg Arg Leu Gln Asn Leu Leu Ser | |
| 20 25 30 | |
| Gly Pro Trp Pro Lys Gln Pro Met Gln Leu His Asn Leu Ser Ser Pro | |
| 35 40 45 | |
| Ser Pro Ser Leu Ser Ser Ser Val Leu Pro Pro Ser Phe Ser Pro Ser | |
| 50 55 60 | |
| Pro Ser Ser Ala Pro Ser Ala Phe Thr Thr Val Gly Gly Ser Ser Gly | |
| 65 70 75 80 | |
| Gly Pro Cys His Pro Thr Ser Ser Ser Leu Val Ser Ala Phe Leu Ala | |
| 85 90 95 | |
| Pro Ile Leu Ala Leu Gln Phe Val Leu Gly Leu Val Gly Asn Ser Leu | |
| 100 105 110 | |
| Ala Leu Phe Ile Phe Cys Ile His Thr Arg Pro Trp Thr Ser Asn Thr | |
| 115 120 125 | |
| Val Phe Leu Val Ser Leu Val Ala Ala Asp Phe Leu Leu Ile Ser Asn | |
| 130 135 140 | |
| Leu Pro Leu Arg Val Asp Tyr Tyr Leu Leu His Gln Thr Trp Arg Phe | |
| 145 150 155 160 | |
| Gly Ala Ala Ala Cys Lys Val Asn Leu Phe Met Leu Ser Thr Asn Arg | |
| 165 170 175 | |
| Thr Ala Ser Val Val Phe Leu Thr Ala Ile Ala Leu Asn Arg Tyr Leu | |
| 180 185 190 | |
| Lys Val Val Gln Pro His His Val Leu Ser Arg Ala Ser Val Gly Ala | |
| 195 200 205 | |
| Ala Ala Arg Val Ala Gly Gly Leu Trp Val Gly Ile Leu Leu Leu Asn | |
| 210 215 220 | |
| Gly His Leu Leu Leu Ser Thr Phe Ser Gly Pro Ser Cys Leu Ser Tyr | |
| 225 230 235 240 | |
| Arg Val Gly Thr Lys Pro Ser Ala Ser Leu Arg Trp His Gln Ala Leu | |
| 245 250 255 | |
| Tyr Leu Leu Glu Phe Phe Leu Pro Leu Ala Leu Ile Leu Phe Ala Ile | |
| 260 265 270 | |
| Val Ser Ile Gly Leu Thr Ile Arg Asn Arg Gly Leu Gly Gly Gln Ala | |
| 275 280 285 | |
| Gly Pro Gln Arg Ala Met Arg Val Leu Ala Met Val Val Ala Val Tyr | |
| 290 295 300 | |
| Thr Ile Cys Phe Leu Pro Ser Ile Ile Phe Gly Met Ala Ser Met Val | |
| 305 310 315 320 | |
| Ala Phe Trp Leu Ser Ala Cys Arg Ser Leu Asp Leu Cys Thr Gln Leu | |
| 325 330 335 | |
| Phe His Gly Ser Leu Ala Phe Thr Tyr Leu Asn Ser Val Leu Asp Pro | |
| 340 345 350 | |
| Val Leu Tyr Cys Phe Ser Ser Pro Asn Phe Leu His Gln Ser Arg Ala | |
| 355 360 365 | |
| Leu Leu Gly Leu Thr Arg Gly Arg Gln Gly Pro Val Ser Asp Glu Ser | |
| 370 375 380 | |
| Ser Tyr Gln Pro Ser Arg Gln Trp Arg Tyr Arg Glu Ala Ser Arg Lys | |
| 385 390 395 400 | |
| Ala Glu Ala Ile Gly Lys Val Lys Val Gln Gly Glu Val Ser Leu Glu | |
| 405 410 415 | |
| Lys Glu Gly Ser Ser Gln Gly | |
| 420 | |
| SEQ ID NO: 3: | |
| cagtggctgc gagaatgctg atg | |
| SEQ ID NO: 4: | |
| gggaatgcca tcctggacac | |
| SEQ ID NO: 5: | |
| caugagaccu ggcgcuuugu uucaccuacc ucaacagugu uuggcgaggu cucucuggaa | 60 |
| auucaaaguc aaccucuuca uguu | 84 |
| SEQ ID NO: 6: | |
| tcttcatctt ctgcatccac acg | |
| SEQ ID NO: 7: | |
| agtggcagga agaactccag cag | |

What is claimed is:

1. A method of decreasing the survival of cancer cells in a mammal, comprising:
providing a compound that interferes directly or indirectly with receptor-mediated action in a cancer cell which otherwise results in prolonged survival of the cancer cell, and
administering the compound to the mammal in a dosage protocol that decreases the survival of the cell,
wherein the receptor comprises a G protein-coupled receptor capable of binding 5-HETE or 5-oxoETE.

2. The method of claim 1, wherein the compound comprises siRNA.

3. The method of claim 1, wherein the mammal is a human.

4. The method of claim 4, wherein the cancer comprises prostate cancer.

5. The method of claim 1, wherein the receptor comprises a polypeptide of SEQ ID NO: 1.

6. A method of treating cancer in a mammal, comprising:
providing a compound that interferes directly or indirectly with receptor-mediated action in a cancer cell which otherwise results in prolonged survival of the cancer cell, and
administering the compound to the mammal in a dosage protocol that decreases the survival of the cell,
wherein the receptor comprised a G protein-coupled receptor capable of binding 5-HETE or 5-oxoETE.

7. The method of claim 6, wherein the compound comprises siRNA.

8. The method of claim 6, wherein the mammal is a human.

9. The method of claim 6, wherein the cancer comprises prostate cancer.

10. The method of claim 6, wherein the receptor comprises a polypeptide of SEQ ID NO: 1.

11. Use in a medicament of a compound that interferes directly or indirectly with receptor-mediated action in a cancer cell which otherwise results in prolonged survival of the cancer cell, wherein the receptor comprises a G-protein coupled receptor capable of binding 5-HETE or 5-oxoETE.

12. The use of claim 11, wherein the receptor is a human prostate cancer cell receptor capable of binding 5-HETE or 5-oxoETE.

13. A method of inducing apoptosis in a mammalian prostate cancer cell, comprising the step of administering a compound to the cells that inhibits expression of a G protein-coupled receptor capable of binding 5-HETE or 5-oxoETE.

14. A method of evaluating whether a compound induces apoptosis in mammalian prostate cancer cells, comprising the steps of:
administering a compound to mammalian prostate cancer cells,
determining whether the compound binds to a G protein-coupled receptor capable of binding 5-HETE or 5-oxoETE, and
determining whether any of the cells undergo apoptosis after administration of the compound.

15. A method of evaluating whether a compound inhibits the expression of a G protein-coupled receptor capable of binding 5-HETE or 5-oxoETE, comprising the steps of:
providing mammalian prostate cancer cells that express a G protein-coupled receptor capable of binding 5-HETE or 5-oxoETE,
determining the level of expression of the receptor in the cells before administration of a test compound;
administering a compound to the cells; and
determining whether the level of expression of the receptor is decreased in the cells after administration of the compound.

16. A polypeptide comprising an amino acid sequence of SEQ ID NO: 1.

17. A polypeptide or comprising an amino acid sequence of SEQ ID NO: 1 in which one or several amino acids are deleted, substituted or added.

18. The polypeptide of claim 16 or claim 17, further comprising a human polypeptide.

19. A nucleic acid which encodes the polypeptide of claim 16 comprising a nucleotide sequence shown by SEQ ID NO: 2.

20. The nucleic acid of claim 19, further comprising a human nucleic acid.

21. A recombinant vector comprising the nucleic acid of claim 19.

22. A host cell into which the recombinant vector of claim 21 is introduced.

23. A method for screening or identifying an agonist or an antagonist of the polypeptide of claim 16, comprising the steps of:
bringing the polypeptide into contact with a test compound, and
detecting in the presence of the test compound, specific binding between the polypeptide and the test compound, or intracellular signal transduction.

24. A method for screening or identifying an antagonist of the polypeptide of claim 16, comprising the steps of:
bringing the polypeptide into contact with a test compound and a ligand;
detecting the function or the activity of the polypeptide; and
determining whether or not the test compound has an ability to inhibit the function or the activity of the polypeptide.

25. The method of claim 24, wherein the function or the activity of the polypeptide is specific binding to a ligand or induction of intracellular signal transductions based on stimulation by a ligand.

26. The method of claim 25, wherein the ligand is 5-oxoETE.

27. The method of claim 25 or claim 26, wherein the polypeptide is in a form of a membrane fraction containing the polypeptide or in a form of a cell expressing the polypeptide on the cell surface.

28. The method of claim 27, wherein the cell expressing the polypeptide on the cell surface is a cell overexpressing the polypeptide by introducing a nucleic acid encoding the polypeptide or by introducing an expression vector comprising the same.

29. A method for inhibiting a function or an activity of the polypeptide of claim 16 in a cell, comprising the step of bringing an antagonist of the polypeptide of claim 16 into contact with a cell expressing the polypeptide of claim 16.

30. A method of decreasing proliferation of prostate cancer cells, or of inducing apoptosis of prostate cancer cells, said method comprising the step of contacting a sample comprising prostate cancer cells with a compound under conditions effective to inhibit binding of 5-HETE or 5-oxoETE to the polypeptide of claim 16.

31. The method of claim 30, wherein the compound inhibits binding of 5-HETE or 5-oxoETE to the polypeptide by binding to the polypeptide.


SEQUENCE LISTING (7 sequences)

SEQ ID NO: 1 - DNA, 1272 bp, Homo sapiens
atgttgtgtcaccgtggtggccagctgatagtgccaatcatcccactttgccctgagcactcctgcaggggtagaagactccagaaccttctctcaggcccatggcccaagcagcccatggaacttcataacctgagctctccatctccctctctctcctcctctgttctccctccctccttctctccctcaccctcctctgctccctctgcctttaccactgtgggggggtcctctggagggccctgccaccccacctcttcctcgctggtgtctgccttcctggcaccaatcctggccctggagtttgtcctgggcctggtggggaacagtttggccctcttcatcttctgcatccacacgcggccctggacctccaacacggtgttcctggtcagcctggtggccgctgacttcctcctgatcagcaacctgcccctccgcgtggactactacctcctccatgagacctggcgctttggggctgctgcctgcaaagtcaacctcttcatgctgtccaccaaccgcacggccagcgttgtcttcctcacagccatcgcactcaaccgctacctgaaggtggtgcagccccaccacgtgctgagccgtgcttccgtgggggcagctgcccgggtggccgggggactctgggtgggcatcctgctcctcaacgggcacctgctcctgagcaccttctccggcccctcctgcctcagctacagggtgggcacgaagccctcggcctcgctccgctggcaccaggcactgtacctgctggagttcttcctgccactggcgctcatcctctttgctattgtgagcattgggctcaccatccggaaccgtggtctgggcgggcaggcaggcccgcagagggccatgcgtgtgctggccatggtggtggccgtctacaccatctgcttcttgcccagcatcatctttggcatggcttccatggtggctttctggctgtccgcctgccgatccctggacctctgcacacagctcttccatggctccctggccttcacctacctcaacagtgtcctggaccccgtgctctactgcttctctagccccaacttcctccaccagagccgggccttgctgggcctcacgcggggccggcagggcccagtgagcgacgagagctcctaccaaccctccaggcagtggcgctaccgggaggcctctaggaaggcggaggccatagggaaggtgaaagtgcagggcgaggtctctctggaaaaggaaggctcctcccagggctga

SEQ ID NO: 2 - protein, 423 aa, Homo sapiens [sequence omitted]

SEQ ID NO: 3 - DNA, 23 bp, Artificial
  Primer
cagtggctgcgagaatgctgatg

SEQ ID NO: 4 - DNA, 20 bp, Artificial
  Primer
gggaatgccatcctggacac

SEQ ID NO: 5 - RNA, 84 nt, Artificial
  Purchased from Dharmacon, Chicago, IL, current product code M-005741-00, described as siGENOME SMARTpool siRNA against human OXER1
caugagaccuggcgcuuuguuucaccuaccucaacaguguuuggcgaggucucucuggaaauucaaagucaaccucuucauguu

SEQ ID NO: 6 - DNA, 23 bp, Artificial
  Primer
tcttcatcttctgcatccacacg

SEQ ID NO: 7 - DNA, 23 bp, Artificial
  Primer
agtggcaggaagaactccagcag